(19) United States
(12) Patent Application Publication
(10) Pub. No.: US 2007/0027339 A1
(43) Pub. Date: Feb. 1, 2007

(54) Ester synthesis

(76) Inventor: William Fullerton, East Riding of Yorkshire (GB)

Correspondence Address: NIXON & VANDERHYE, PC, 901 NORTH GLEBE ROAD, 11TH FLOOR, ARLINGTON, VA, 22203, US

(21) Appl. No.: 10/569,440

(22) PCT Filed: Aug. 24, 2004
(86) PCT No.: PCT/GB04/03619

§ 371 Date: Feb. 24, 2006

(30) Foreign Application Priority Data
Sep. 3, 2003 (GB) 0320692.7

(51) Int. Cl. C07C 67/05

(52) U.S. Cl. 560/243

(57) ABSTRACT

Process for making lower aliphatic ester, especially ethyl acetate, by reacting a lower olefin with a saturated lower aliphatic mono-carboxylic acid in the vapor phase using a heteropolyacid catalyst, wherein the reaction pressure is 11 to 20 barg (1100 to 2000 KPa), more preferably 12 to 15 barg (1200 to 1500 KPa). The reaction temperature is 140 to 250° C., more preferably 160 to 195° C. The process reduces the level of by-products, for example methyl ethyl ketone and/or acetaldehyde.

The present invention relates to a process for the synthesis of esters by reacting an olefin with a lower carboxylic acid in the presence of an acidic catalyst.

It is well known that olefins can be reacted with lower aliphatic carboxylic acids to form the corresponding esters. One such method is described in GB-A-1259390 in which an ethylenically unsaturated compound is contacted with a liquid medium comprising a carboxylic acid and a free heteropolyacid of molybdenum or tungsten. This process is a homogeneous process in which the heteropolyacid catalyst is unsupported. A further process for producing esters is described in JP-A-05294894 in which a lower fatty acid is reacted with a lower olefin to form a lower fatty acid ester. In this document, the reaction is carried out in the gaseous phase in the presence of a catalyst consisting of at least one heteropolyacid salt of a metal e.g. Li, Cu, Mg or K, being supported on a carrier. The heteropolyacid used is phosphotungstic acid and the carrier described is silica.

EP-A-0757027 (BP Chemicals) discloses a process for the production of lower aliphatic esters, for example ethyl acetate, by reacting a lower olefin with a saturated lower aliphatic carboxylic acid in the vapour phase in the presence of a heteropolyacid catalyst characterised in that an amount of water in the range from 1-10 mole % based on the total of the olefin, aliphatic mono-carboxylic acid and water is added to the reaction mixture during the reaction. The presence of water is said to reduce the amount of unwanted by-products generated by the reaction.

The reaction disclosed in the prior art can be carried out, for example, at pressures in the range 400-3000 KPa (4-30 barg), preferably 500-3000 KPa (5-30 barg). The pressure employed in the processes disclosed in all the Examples of EP-A-0757127 is 1000 KPa (10 barg).

A general problem encountered with the above processes for the production of esters using heteropolyacid catalysts is the generation of small amounts of a variety of by-products. These by-products generally have to be removed from the ester product by separation processes such as fractional distillation and solvent extraction.

It is an object of the present invention to provide an improved process for the production of lower aliphatic esters by reacting an olefin with lower aliphatic carboxylic acid in the presence of heteropolyacid catalyst. It is a further object to provide a process for the production of lower aliphatic esters by reacting an olefin with a lower aliphatic carboxylic acid in the presence of heteropolyacid catalyst wherein there is a reduced production of undesirable by-products.

Accordingly, the present invention is a process for the production of a lower aliphatic ester, said process comprising reacting a lower olefin with a saturated lower aliphatic mono-carboxylic acid in the vapour phase in the presence of a heteropolyacid catalyst, characterised in that the reaction pressure employed lies in the range 11 to 20 barg (1100 to 2000 KPa), preferably in the range 12 to 18 barg (1200 to 1800 KPa), more preferably in the range 12 to 15 barg (1200 to 1500 KPa).

The process of the present invention surprisingly provides a reduction in the generation of at least some undesirable impurities, for example, aldehydes, ketones and a variety of saturated and unsaturated hydrocarbon species of carbon chain length varying, for example, from C₆ to C₂₀₊, including polycyclic aromatic ring containing hydrocarbons. In particular, in the production of ethyl acetate from ethylene and acetic acid, operation of the process at pressures in the defined range results in a substantial reduction in the production of certain volatile by-products, especially butan-2-one (commonly know as “methyl ethyl ketone ” or “MEK”), and acetaldehyde, without adversely affecting the production of the desired ester.

The invention further provides a process for the production of ethyl acetate by reacting ethylene with acetic acid in the presence of a heteropolyacid catalyst at a temperature in the range 140 to 250° C., preferably 150 to 240° C., mote preferably 160 to 195° C. wherein the reaction pressure is maintained in the range 11 to 20 barg (1100 to 2000 KPa), preferably in the range 12 to 15 barg (1200 to 1500 KPa) to reduce the level of by-product methyl ethyl ketone and/or acetaldehyde in the reaction product.

The term “heteropolyacid” as used herein and throughout the specification is meant to include the free acids and/or metal salts thereof. The heteropolyacids used to prepare the esterification catalysts of the present invention therefore include inter alia the free acids and co-ordination type salts thereof in which the anion is a complex, high molecular weight entity. The heteropolyacid anion comprises from two to eighteen oxygen-linked polyvalent metal atoms, which are generally known as the “peripheral” atoms. These peripheral atoms surround one or more central atoms in a symmetrical manner. The peripheral atoms are usually one or more of molybdenum, tungsten, vanadium, niobium, tantalum and other metals. The central atoms are usually silicon or phosphorus but can comprise any one of a large variety of atoms from Groups I-VIII in the Periodic Table of elements. These include, for instance, cupric ions; divalent beryllium, zinc, cobalt or nickel ions; trivalent boron, aluminium, gallium, iron, cerium, arsenic, antimony, phosphorus, bismuth, chromium or rhodium ions; tetravalent silicon, germanium, tin, titanium, zirconium, vanadium, sulphur, tellurium, manganese nickel, platinum, thorium, hafnium, cerium ions and other rare earth ions; pentavalent phosphorus, arsenic, vanadium, antimony ions; hexavalent tellurium ions; and heptavalent iodine ions. Such heteropolyacids are also known as “polyoxoanions”, “polyoxometallates” or “metal oxide clusters”.

Heteropolyacids usually have a high molecular weight e.g. in the range from 700-8500 and include dimeric complexes. They have a relatively high solubility in polar solvents such as water or other oxygenated solvents, especially if they are free acids and in the case of several salts, and their solubility can be controlled by choosing the appropriate counter-ions. Specific examples of heteropolyacids and their salts that may be used as the catalysts in the present invention include:

| | |
|---|---|
| 12-tungstophosphoric acid | H₃[PW₁₂O₄₀].xH₂O |
| 12-molybdophosphoric acid | H₃[PMo₁₂O₄₀].xH₂O |
| 12-tungstosilicic acid | H₄[SiW₁₂O₄₀].xH₂O |
| 12-molybdosilicic acid | H₄[SiMo₁₂O₄₀].xH₂O |
| Cesium hydrogen tungstosilicate | Cs₃H[SiW₁₂O₄₀].xH₂O |
| Potassium tungstophosphate | K₆[P₂W₁₈O₆₂].xH₂O |
| Ammonium molybdodiphosphate | (NH₄)₆[P₂Mo₁₈O₆₂].xH₂O |

Preferred heteropolyacid catalysts for use in the present invention are tungstosilicic acid and tungstophosphoric acid. Particularly preferred are the Keggin or Wells-Dawson or Anderson-Evans-Perloff primary structures of tungstosilicic acid and tungstophosphoric acid.

The heteropolyacid catalyst whether used as a free acid or as a salt thereof can be supported or unsupported. Preferably the heteropolyacid is supported. Examples of suitable supports are relatively inert minerals with either acidic or neutral characteristics, for example, silicas, clays, zeolites, ion exchange resins and active carbon supports. Silica is a particularly preferred support. When a support is employed, it is preferably in a form which permits easy access of the reactants to the support. The support, if employed, can be, for example, granular, pelletised, extruded or in another suitable shaped physical form. The support suitably has a pore volume in the range from 0.3-1.8 ml/g, preferably from 0.6-1.2 ml/g and a crush strength of at least 7 Kg force. The crush strengths quoted are based on average of that determined for each set of 50 particles on a CHATTILLON tester which measures the minimum force necessary to crush a particle between parallel plates. The support suitably has an average pore radius (prior to supporting the catalyst thereon) of 10 to 500 Å preferably an average pore radius of 30 to 150 Å.

In order to achieve optimum performance, the support is suitably free from extraneous metals or elements which can adversely affect the catalytic activity of the system. If silica is employed as the sole support material it preferably has a purity of at least 99% w/w, i.e. the impurities are less than 1% w/w, preferably less than 0.60% w/w and more preferably less than 0.30% w/w.

Preferably the support is derived from natural or synthetic amorphous silica. Suitable types of silica can be manufactured, for example, by a gas phase reaction, (e.g. vaporisation of SiO₂ in an electric arc, oxidation of gaseous SiC, or flame hydrolysis of SiH₄ or SiCl₄), by precipitation from aqueous silicate solutions, or by gelling of silicic acid colloids. Preferably the support has an average particle diameter of 2 to 10 mm, preferably 4 to 6 mm. Examples of commercially available silica supports that can be employed in the process of the present invention are Grace 57 granular and Grace SMR 0-57-015 extrudate grades of silica. Grace 57 silica has an average pore volume of about 1.15 ml/g and an average particle size ranging from about 3.0-6.0 mm.

The impregnated support can be prepared by dissolving the heteropolyacid, in e.g. distilled or demineralised water, and then adding the aqueous solution so formed to the support. The support is suitably left to soak in the acid solution for a duration of several hours, with periodic manual stirring, after which time it is suitably filtered using a Buchner funnel in order to remove any excess acid.

The wet catalyst thus formed is then suitably placed in an oven at elevated temperature for several hours to dry, after which time it is allowed to cool to ambient temperature in a desiccator. The weight of the catalyst on drying, the weight of the support used and the weight of the acid on support were obtained by deducting the latter from the former from which the catalyst loading in g/litre was determined.

Alternatively, the support may be impregnated with the catalyst using by spraying a solution of the heteropolyacid on to the support with simultaneous or subsequent drying (eg in a rotary evaporator).

This supported catalyst can then be used in the esterification process. The amount of heteropolyacid deposited/impregnated on the support for use in the esterification reaction is suitably in the range from 10 to 60% by weight, preferably from 30 to 50% by weight based on the total weight of the heteropolyacid and the support.

In the reaction, the olefin reactant used is preferably ethylene, propylene or mixtures thereof. Where a mixture of olefins is used, the resultant product will be inevitably a mixture of esters. The source of the olefin reactant used may be a refinery product or a chemical or a polymer grade olefin which may contain some alkanes admixed therewith. Most preferably the olefin is ethylene.

The saturated, lower aliphatic mono-carboxylic acid reactant is suitably a C₁-C₄ carboxylic acid and is preferably acetic acid.

Preferably the reactants fed or recycled to the reactor contain less than 1 ppm, most preferably less than 0.1 ppm of metals, or metallic compound or basic nitrogen (eg ammonia or amine) impurities. Such impurities can build up in the catalyst and cause deactivation thereof.

The reaction mixture suitably comprises a molar excess of the olefin reactant with respect to the aliphatic mono-carboxylic acid reactant. Thus the mole ratio of olefin to the lower carboxylic acid in the reaction mixture is suitably in the range from 1:1 to 15:1, preferably from 10:1 to 14:1.

The reaction is carried out in the vapour phase suitably above the dew point of the reactor contents comprising the reactant acid, any alcohol formed in situ, the product ester. It is preferred to use at least some water in the reaction mixture. The amount of water can be, for example, in the range from 1-10 mole %, preferably from 1-7 mole %, more preferably from (1-5 mole %) based on the total amount of olefin, carboxylic acid and water. The meaning of the term “dew point” is well known in the art, and is essentially, the highest temperature for a given composition, at a given pressure, at which liquid can still exist in the mixture. The dew point of any vaporous sample will thus depend upon its composition.

The supported heteropolyacid catalyst is suitably used as a fixed bed which may be in the form of a packed column, or radial bed or a similar commercially available reactor design. The vapours of the reactant olefins and acids are passed over the catalyst suitably at a GHSV in the range from 100 to 5000 per hour, preferably from 300 to 2000 per hour.

The reaction is suitably carried out at a temperature in the range from 150-200° C. The reaction pressure, as stated previously, is in the range 11 to 20 barg, preferably from 12 to 15 barg.

The water preferably added to the reaction mixture is suitably present in the form of steam and is capable of generating a mixture of esters and alcohols in the process. The products of the reaction are recovered by e.g. fractional distillation. Where esters are produced, whether singly or as mixture of esters, these may be hydrolysed to the corresponding alcohols or mixture of alcohols in relatively high yields and purity. By using this latter technique the efficiency of the process to produce alcohols from olefins is significantly improved over the conventional process of producing alcohols by hydration of olefins.

The invention is now illustrated in the following Examples and accompanying drawings.

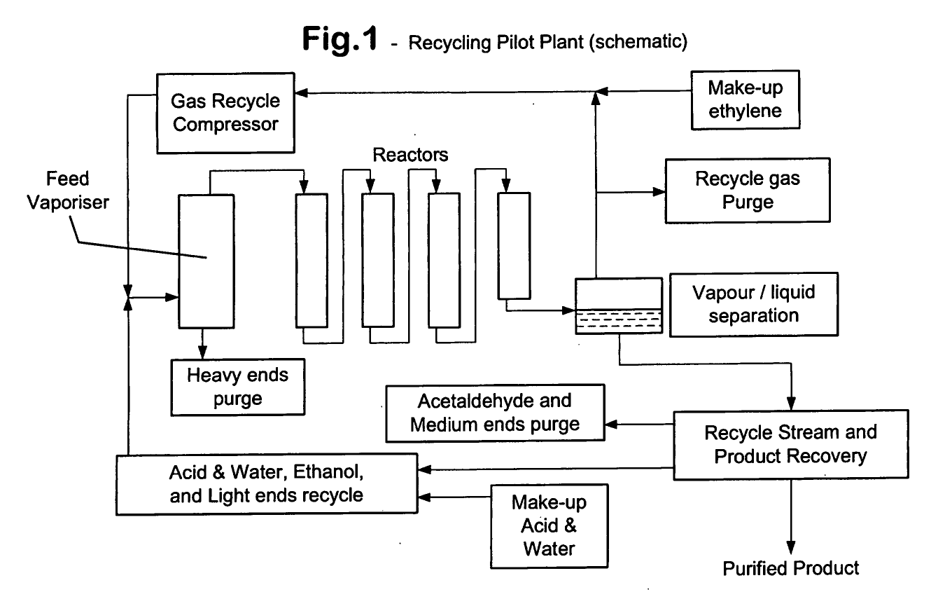

FIG. 1 represents diagrammatically a pilot plant scale apparatus for the manufacture of ethyl acetate;

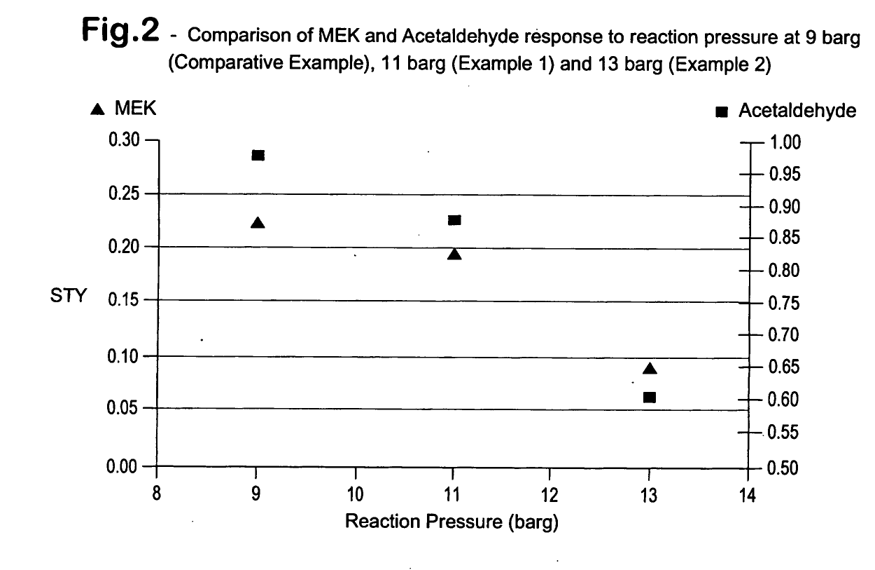

FIGS. 2-4 show graphically quantities of impurities produced in the reaction of ethylene with acetic acid at various pressures.

### EXAMPLES 1-3

Examples 1 and 2 are in accordance with the present invention and Example 3 is by way of comparison. The following Examples were performed in a demonstration plant incorporating feed, reaction and product recovery sections, including recycle of the major by-product streams and known as a “fully recycling pilot plant”. An outline description of the layout-and mode of operation of this equipment is given below.

Catalyst productivity towards some components is reported in STY units, (defined as grams of quoted component per litre of catalyst per hour).

Recycling Pilot Plant-Description

The apparatus used to generate these Examples was an integrated recycle pilot plant designed to mimic the operation of a 220 kte commercial plant at an approximate scale of 1:7000.

A basic flow diagram of the unit is shown in FIG. 1. The unit comprises a feed section (incorporating a recycle system for both unreacted feeds and all the major by-products), a reaction section, and a product and by-product separation section. The feed section utilises liquid feed pumps to deliver fresh acetic acid, fresh water, unreacted acid/water, ethanol and light ends recycle streams to a vapouriser. The ethylene feed also enters the vapouriser where it is premixed with the liquid feeds. The -ethylene is fed both as a make-up stream, but more predominantly as a recycle stream and is circulated around the system at a desired rate and ethylene content. The combined feed vapour stream is fed to a reactor train; comprising four fixed bed reactors, each containing a 5 litre catalyst charge.

The first three reactors are fitted with acid/water injection to the exit streams to facilitate independent control of reactor inlet temperatures.

The crude product stream exiting the reactors is cooled before entering a flash vessel where the separation of non-condensable (gas) and condensable (liquid) phases occurs. The recovered gas is recycled back to the vapouriser with the exception of small bleed stream removed to assist control of recycle stream purity. The liquid stream enters the product separation and purification system, which is a series of distillation columns designed to recover and purify the final product and also to recover the unreacted acetic acid, water, ethanol and light ends streams for recycling back to the vapouriser. Small bleed streams located in the liquid recovery enable the removal of undesired recycle components from the process during this stage.

Analysis and Reporting

The sample points for analysis in the Examples were as follows; The ethyl acetate production reported is recorded at point (a) and calculated using Coriolis meter mass flow measurement and Near Infrared (NIR) analysis of the crude liquid stream composition, calibrated in wt %.

The reported figures for MEK and acetaldehyde production are recorded on the residual crude product after the acid/water recycle stream has been separated. The stream composition is measured using an Agilent model 6890 gas liquid chromatograph equipped with both FID and TCD detectors to determine both major (wt %) and minor (μm) components. The fitted column is a 60 m×0.32 mm i.d. DB1701 with a 1 μm film thickness operated on Helium carrier gas flow of 2 ml min⁻¹ and split ratio of 25:1. The sampling system employed is an online closed loop system, with continuous sample flushing. The STY value for these components has been calculated from the reported concentrations and expressed with respect to ethyl acetate STY.

The reported hydrocarbon analysis is from a sample of recycle light ends feed analysed offline using a Chrompack CP9001 gas chromatograph equipped with and-FID detector. The fitted column is a 50 m×0.32 mm i.d. CP Sil 8 with a 1.2 μm film thickness operated on Helium carrier gas flow of 2 ml mind⁻¹ and split ratio of 20:1. The quoted components were identified by GCMS.

Experimental Conditions

The catalyst employed was 12-tungstosilicic heteropolyacid supported on Grace 57 silica at a catalyst loading of 140 grams per litre.

The experiment involved start-up and initial operation within standard parameters to obtain a steady baseline activity and impurity make rates. The total system pressure was then varied, by adjusting the recycle compressor discharge pressure, while maintaining other variables constant. The shutdown involved taking off feeds, reducing system pressure to atmospheric, and cooling the unit to ambient temperature, using a standard operating procedure designed to protect the catalyst. A summary of the key operating conditions and results is given in Table 1.

TABLE 1
Experimental conditions and results

| | Example No. | | |
|---|---|---|---|
| | 1 | 2 | Comp 3 |
| Pressure (barg) | 11 | 13 | 9 |
| Ethylene:acetic acid | 11.1 | 11.1 | 11.1 |
| Acetic acid (mol %) | 7.1 | 7.1 | 7.1 |
| Water (mol %) | 5.1 | 5.1 | 5.1 |
| Recycle gas rate (kg/hr) | 26.0 | 26.0 | 26.0 |
| Recycle gas purity (wt % C₂⁻) | 90.0 | 90.0 | 90.0 |
| Average Reactor Inlet Temperature (° C.) | 172 | 172 | 172 |
| Etac STY (g/litre catalyst/hr) | 200 | 199 | 199 |
| Diethyl ether STY (g/litre catalyst/hr) | 3.64 | 3.38 | 3.40 |
| Ethanol STY (g/litre catalyst/hr) | 7.84 | 7.95 | 7.56 |
| MEK STY (g/litre catalyst/hr) | 0.195 | 0.087 | 0.224 |
| Acetaldehyde STY (g/litre catalyst/hr) | 0.874 | 0.598 | 0.974 |

Results

As can be noted from Table 1, the effect of varying pressure over the experimental range had negligible impact on catalyst productivity of ethyl acetate at a constant reactor inlet temperature. The effect of pressure was also noted to be minor towards the make-rates of the major by-products in the process; namely ethanol and diethyl ether.

It will be noted that operation at 9 barg (Comparative Example 3) provides relatively high make-rates of both MEK and acetaldehyde, while operation in accordance with the present invention at, respectively 11 and 13 barg (Examples 1 and 2), resulted in significant decrease in the concentrations of both these by-products. The response to pressure of these materials is displayed on FIG. 2 of the Drawings.

The make rates of a variety of other minor reaction by-products were also observed to change as a result of changes in the reaction pressure.

The reaction produces a range of hydrocarbon impurities at similar levels, at concentrations of up to 1000 ppm in the crude product stream. These impurities range mainly from C₄ to C₈ carbon numbers in chain length. However, they can grow in chain length up to C₂₊ upon recycle through the reactor train.

These hydrocarbons may take the forms of saturated or unsaturated, branched or linear species; i.e. 2-methylpentane, 3-methylpentane, 2-methylhexane, 2,3-dimethylpentane, 3-methylhexane, trimethylpent-2-ene, and 2-methyl-2-heptene, have all been identified as well as many other analogous species.

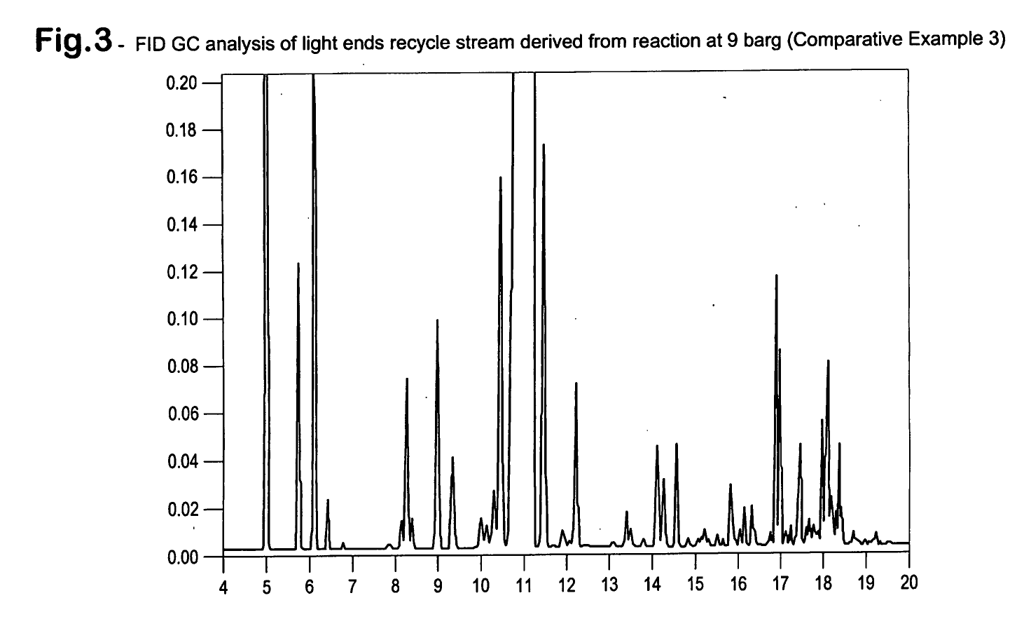

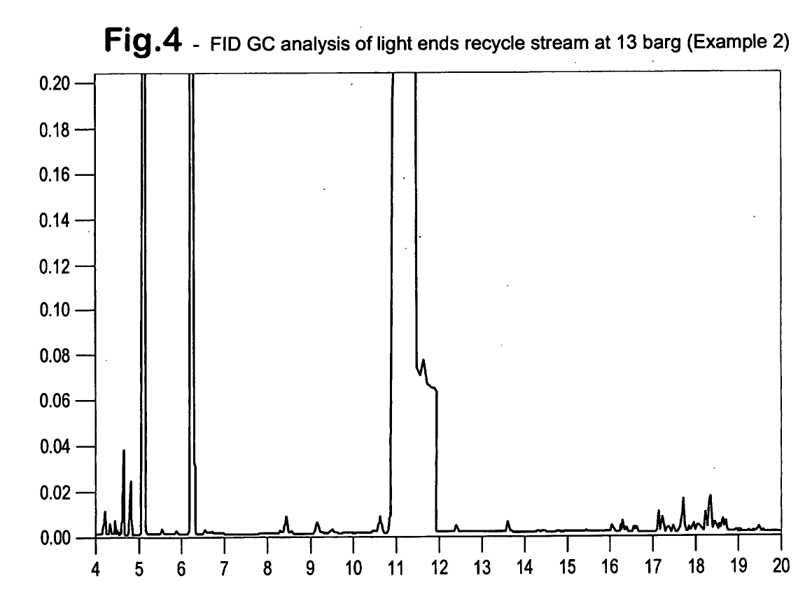

In comparing analysis of the 9 barg and 13 barg operation product streams, by FID gas chromatography, it is noted that the reduction in these by-products is significant. In the majority of cases, the measured component level at the higher pressure operation represents only 10% of that obtained at the lower pressure, and in some cases, as low as 1%. This difference is illustrated by comparison of FIGS. 3 and 4 which show Gas Chromatograms of the crude product streams.

Significant reduction of other oxygenated hydrocarbon by-products also occurs at 13 barg operation, including but not limited to; acetone (reduced by 90%), ethyl formate (reduced by 90%), 3-pentanone (reduced by 90%), and ethyl propionate (reduced by 50%). Not all of the process impurities in the stream have been identified.

The heavier hydrocarbon species, up to C₂₀₊, also undergo significant overall reduction at higher pressure, being measured at 40% of the lower pressure value, also by FID gas chromatography, although no distinction is made between the individual components in this measurement.

As the aforementioned impurities predominantly originate from an ethylene precursor, the operation of the process at higher pressure improves the catalyst selectivity based on ethylene by inhibiting the formation of these species. Since the process must typically remove the majority of these components by means of a purge stream, the benefit of higher pressure operation will allow process operation with significant reduction or elimination of some or all of these purge streams. It is reasonable to suppose that further increases in pressure could extend the benefit further.

The reductions in acetaldehyde and methyl ethyl ketone for example enable extended catalyst life as this material has previously been identified as a catalyst deactivation precursor. Similarly 2-butanone. The hydrocarbon species will also play a role in catalyst deactivation by providing a source of coke for the catalyst surface and hence providing a barrier between the reactants and the catalyst active sites as coke formation increases. It is therefore believed that significant reduction of these species will allow extension of catalyst life and deliver commercial benefit.

### EXAMPLE 4 AND COMPARATIVE EXAMPLE 5

The data for these Examples was collected on a catalyst development microreactor. The microreactor is a single pass tubular reactor holding 6.25 ml of silicotungstic acid on silica catalyst ground to 0.5-1 mm particle size mixed with 6.25 ml silica 0.5-1 mm particle size. The reactor was a tubular gas phase downward flow reactor. Standard feed conditions used were 23.81 g/hr ethylene, 3.65 ml/hr acetic acid, 1 m/hr water and 0.54 ml/hr diethyl ether additionally 1% w/w 2-butanol were doped into the liquid feed as a by-product precursor. The reactor was heated to 185° C., the liquid and gas components were fed into the reactor over a 60ml carborundum pre-heat bed to ensure full vaporisation and mixing of the liquid components with the gas. The pre-heat bed were separated from the catalyst using a glass wool plug and the catalyst bed was then supported on a further glass wool plug. Under standard running conditions the pressure was maintained at 10 barg with a gas hourly space velocity of 3600. The products from the reactor were cooled and the liquid components were collected and analysed by liquid GC, the gas components were analysed by an online refinery gas GC.

In these Examples the reactor was started up under the standard conditions described above. After 110 HOS (hours on stream) the catalyst had bedded in and was producing steady data. At this point the acetaldehyde make of the catalyst was 0.24 g/lcat/hr and the methylethylketone make was 0.011 g/lcat/hr. After the samples were taken the reactor pressure was increased to 12.9 barg, all other parameters, feed rate, reactor temperature etc were kept the same. After 132 HOS the acetaldehyde make had reduce to 0.14 g/lcat/hr and the methylethylketone make had decreased to 0.007 g/lcat/hr. The results are shown in Table 2.

TABLE 2

| | | | Acetaldehyde | |
|---|---|---|---|---|
| | | Pressure | make | Methylethylketone |
| Example | HOS | (barg) | (g/lcat/h) | (g/lcat/hr) |
| Comp. 5 | 110 | 10 | 0.24 | 0.011 |
| 4 | 132 | 12.9 | 0.14 | 0.007 |

1-20. (canceled)

21. A process for the production of a lower aliphatic ester comprising reacting a lower olefin with a saturated lower aliphatic mono-carboxylic acid in the vapour phase in the presence of a heteropolyacid catalyst, characterised in that the reaction pressure employed lies in the range 12 to 18 barg (1300 to 1900 KPa).

22. A process as claimed in claim 21 characterised in that the reaction pressure employed lies in the range preferably in the range 12 to 15 barg (1300 to 1600 KPa).

23. A process for the production of ethyl acetate by reacting ethylene with acetic acid in the presence of a heteropolyacid catalyst at a temperature in the range 140 to 250° C., wherein the reaction pressure is maintained in the range 12 to 15 barg (1300 to 1600 Kpa).

24. A process as claimed in claim 21 wherein the heteropolyacid is selected from 12-tungstophosphoric acid, 12-molybdophosphoric acid, 12-tungstosilicic acid and 12-molybdosilicic acid.

25. A process as claimed in claim 21 or claim 23 wherein the heteropolyacid is supported.

26. A process as claimed in claim 25 wherein the support is selected from silica, clay, zeolite, ion exchange resins and active carbon.

27. A process as claimed in claim 26 wherein the support is derived from natural or synthetic amorphous silica.

28. A process as claimed in claim 26 or 27 wherein the support is made by flame hydrolysis of SiH₄ or SiCl₄.

29. A process as claimed in claim 26 or 27 wherein the support is made by precipitation from aqueous silicate solution, or by gelling of silicic acid colloids.

30. A process as claimed in claim 26 wherein the support has an average particle diameter of 4 to 6 mm.

31. A process as clamed in claim 21 or 22 wherein the olefin is ethylene, propylene or mixtures thereof.

32. A process as claimed in claim 21 or 22 wherein the saturated, lower aliphatic mono-carboxylic acid reactant is a C₁-C₄ carboxylic acid.

33. A process as claimed in claim 21 or 22 wherein the saturated, lower aliphatic mono-carboxylic acid reactant is acetic acid.

34. A process as claimed in claim 21 or 22 wherein the mole ratio of olefin to the lower carboxylic acid in the reaction mixture is in the range from 1:1 to 15:1.

35. A process as claimed in claim 34 wherein the mole ratio of olefin to the lower carboxylic acid in the reaction mixture in the range from 10:1 to 14:1.

36. A process as claimed in claim 21 or 22 wherein at least some water is used in the reaction mixture.

37. A process as claimed in claim 36 wherein the amount of water is in the range from 1-10 mole % based on the total amount of olefin, carboxylic acid and water.

38. A process as claimed in claim 23 wherein the mole ratio of olefin to the lower carboxylic acid in the reaction mixture is in the range from 1:1 to 15:1.

39. A process as claimed in claim 38 wherein the mole ratio of olefin to the lower carboxylic acid in the reaction mixture in the range from 10:1 to 14:1.

40. A process as claimed in claim 23 wherein at least some water is used in the reaction mixture.

41. A process as claimed in claim 40 wherein the amount of water is in the range from 1-10 mole % based on the total amount of olefin, carboxylic acid and water.
